(19) United States
(12) Patent Application Publication
(10) Pub. No.: US 2024/0122525 A1
(43) Pub. Date: Apr. 18, 2024

(54) Spinal-Cord Stimulation Techniques for Predicting Current or Future Myelination and/or Objectively Characterizing Multiple Sclerosis State

(71) Applicant: Rune Labs, Inc., San Francisco, CA (US)

(72) Inventors: Ro’ee Gilron, Fairfax, CA (US); Teresa Wen, Fairfax, CA (US)

(73) Assignee: Rune Labs, Inc., San Francisco, CA (US)

(21) Appl. No.: 18/478,471

(22) Filed: Sep. 29, 2023

Related U.S. Application Data
(60) Provisional application No. 63/379,405, filed on Oct. 13, 2022.

(51) Int. Cl. A61B 5/00; A61B 5/388

(52) U.S. Cl. CPC A61B 5/407; A61B 5/388; A61B 5/7275

(57) ABSTRACT

Some disclosed techniques relate to extracting one or more features based on an average evoked response and generating a result that identifies: a predicted degree of inflammation; a predicted degree of myelination; a predicted degree of demyelination; or a predicted degree to which a symptom or disease of the subject would be effectively treated by a remyelination therapy. Some disclosed techniques relate to accessing evoked compound action potentials, mapping the evoked potentials to a functional system; generating an average evoked response; extracting one or more features from the average evoked response; and generating a functional-system impairment metric associated with the functional system based on the one or more features, where the functional-system impairment metric indicates whether or an extent to which the functional system of the subject is impaired.

### CLAIM FOR PRIORITY

This application claims priority to U.S. Provisional Patent Application No. 63/379,405, filed on Oct. 13, 2022, titled “SPINAL-CORD STIMULATION TECHNIQUES FOR PREDICTING CURRENT OR FUTURE MYELINATION AND/OR OBJECTIVELY CHARACTERIZING MULTIPLE SCLEROSIS STATE,” and which is incorporated by reference in entirety.

### BACKGROUND

Multiple sclerosis (MS) is a disease that affects 1 in 200 people in the United States. Typically, subjects initially present with the relapse-remitting type of multiple sclerosis, where subjects experience discrete exacerbations (which may be followed by some degree of recovery). During each exacerbation, the subject's immune cells cross the blood-brain barrier and attack the brain and/or spinal cord (referred to as inflammation). More specifically, the immune cells attack myelin (thereby causing demyelination) and/or oligodendrocytes (thereby causing oligodendrocyte injury). During these attacks, subjects may experience symptoms, such as numbness, optic neuritis, poor balance, tingling, etc. Frequently, initial symptoms are sensory and/or visual.

Over time (e.g., within about 10-20 years), most subjects transition from the relapse-remitting type of multiple sclerosis to the secondary progressive type of multiple sclerosis. For example, about 90% of subjects initially diagnosed with relapse-remitting multiple sclerosis transition to the secondary progressive type within 25 years. During this transition, the disease transitions from being an inflammatory disease (where there are discrete immune-system attacks) to being a neurodegenerative disease. That is, neurons' axons are left without a protective myelin sheath and there are too few properly functioning oligodendrocytes to repair the sheath, meaning that the neurons' axons are then prone to degeneration. Axon degeneration leads to death of the neurons and brain atrophy (shrinking of the brain). As neurodegeneration becomes increasingly extensive, disability becomes more severe and increasingly affects motor functions (in addition to other functions).

Though the typical disease course of multiple sclerosis follows this sequence of initial presentation of relapse-remitting multiple sclerosis that transitions to the secondary progressive type, there are other possible disease courses. For example, some subjects spend many decades with the relapse-remitting type and never transition to the secondary progressive type. As another example, some subjects initially present with a primary progressive type of multiple sclerosis (e.g., where there are no discrete exacerbations and instead there is a gradual decline in function and a gradual increase in brain atrophy).

In addition to the variety of disease types and potential disease presentations, there is also very high variability across subjects as to how quickly and/or the extent to which multiple sclerosis progresses (e.g., in general or throughout a given stage) and the extent to which the disease will progress and/or respond to a given therapy. Magnetic resonance imaging (MRI) scans can be used to show inflammation, dark holes, and/or brain atrophy. However, there is not a deterministic relationship between any of these features and the functional capabilities of a subject. For example, a first subject with multiple sclerosis may have 10 times the lesion load as that of a second subject but still have fewer functional deficits.

Further, the medical community often uses a scale to assess multiple sclerosis subjects. A prominent scale is the Expanded Disability Status Scale (EDSS), which is rooted in motor-system capabilities. However, the EDSS is non-linear, bi-modal (with scores towards the middle of the scale being relatively uncommon), and overemphasizes ambulation. Meanwhile, the scale is structured such that cognitive function is not well captured, the inter-rater reliability is low, and scores can be ambiguous in some cases. While EDSS scores are correlated with quality of life, it is difficult to predict EDSS scores based on other variables.

Thus, there has been little success in trying to predict a specific progression course for a subject.

The vast majority of treatments currently available aim to slow the progression of the disease. Some (e.g., a corticosteroids, such as methylprednisolone) are prescribed to treat relapses (e.g., by promoting closure of the blood-brain barrier so as to prevent more immune cells from attacking the central nervous system (and thus reducing inflammation). Still other treatments are used to treat symptoms caused by slower or absent nerve conduction or by lesions caused by multiple sclerosis. Data frequently indicates a high variability concerning the extent to which a given type of treatment effectively treats multiple sclerosis, even when there are controls set with regard to the type of multiple sclerosis, demographics, etc.

Importantly, it can be difficult to even determine how to assess the efficacy of a treatment and/or characterize a state of multiple sclerosis. For example, one approach for characterizing a disease state and/or treatment efficacy may be to characterize an extent to which MS subjects treated with a given therapy decline along the EDSS scale relative to other MS subjects. However—as noted above—this scale is imperfect.

As another example, one approach for characterizing a disease state and/or treatment efficacy may be to characterize an extent to which MS subjects treated with a given therapy have fewer relapses relative to other MS subjects. However, effects of a treatment on relapse rate may be much delayed and may further depend highly on the initial state of the disease.

As yet another example, one approach for characterizing a disease state and/or treatment efficacy may be to characterize MRI results (e.g., an extent of inflammation or lesion load). However, while regions that are enhanced following administration of a contrast agent to a subject may indicate that there is an active lesion (with inflammation), these signals may be transitory, difficult to quantitatively characterize, and only representative of damage associated with damage to the blood-brain barrier. Further, collecting MRI scans with contrast has a risk to subjects, in that the heavy-metal contrast agents that are administered to the subject are not subsequently excreted by the subject, meaning that they build-up over time. Further yet, lesion load may be poorly correlated with a state of the disease.

As still another example, one approach for characterizing a disease state and/or treatment efficacy may include evaluating features of evoked responses. Here, a stimulus is presented (e.g., a visual stimulus presented on a screen, an audio stimulus presented via a speaker, a transcranial magnetic stimulation) to or given to a subject, and one or more electrodes (e.g., an electroencephalography electrode or an electromyography electrode) are used to non-invasively capture a response signal. One or more latency variables are then calculated to characterize a delay between the stimulus and a particular part of the evoked response. The stimulus presentations are typically non-invasively delivered or are delivered in a minutely invasive manner (e.g., when capturing motor evoked responses) that does not require an incision. However, current assessments of evoked responses are constrained in that there is a limited spatial resolution at which the pathology can be characterized. Further, current assessments of evoked responses are not sensitive or specific enough to detect local inflammation, which means that the data may provide little information as to what type of multiple sclerosis a subject has—an inflammatory relapse-remitting type or a neurodegenerative progressive type. That is, current results may be unable to attribute delay in a feature of an evoked response to either demyelination of a nerve or nerve damage.

Thus, there is a need to improve a degree to which a state of a disease (e.g., multiple sclerosis) is objectively and reliably characterized and to improve a degree to which efficacy of a given treatment can be characterized. These objectives can facilitate evaluating a potential treatment, defining indications of a treatment, selecting a particular treatment for a particular subject, etc.

### SUMMARY

In some embodiments, a method is provided that includes: detecting a set of evoked compound action potentials that were measured using one or more electrodes, wherein each of the set of evoked compound action potentials is defined to start at a corresponding stimulation time of a set of stimulation times; generating an average evoked response using the set of evoked compound action potentials; extracting one or more features from the average evoked response; generating a result based on the one or more features, wherein the result identifies: a predicted degree of inflammation; a predicted degree of myelination; a predicted degree of demyelination; or a predicted degree to which a symptom or disease of the subject would be effectively treated by a remyelination therapy; and outputting the result.

Each of the set of evoked compound action potentials may include an antidromic compound action potential. Each of the set of evoked compound action potentials may include a dromic compound action potential. The spinal cord may have been stimulated at each of the set of stimulation times using an electrode positioned via percutaneous access to the spinal cord. The one or more features may include a time of a peak or trough in the average evoked response. The one or more features may include a magnitude or relative magnitude of a peak or trough in the average evoked response. The one or more electrodes may have been positioned on a head of the subject to collect the set of evoked compound action potentials using signals from a brain of the subject. Generating the result may include using a look-up table that associates various feature values with quantitative or qualitative predicted degrees of inflammation or of myelination. Generating the result may include using a function that relates feature values with quantitative or qualitative predicted degrees of inflammation or of myelination. The result may be a category. The method may further include stimulating the spinal cord of the subject using the one or more electrodes at each of the set of stimulation times; and measuring the set of evoked compound action potentials. The result may identify the predicted degree of inflammation. The result may identify the predicted degree of myelination or demyelination. The result may identify the predicted degree to which the symptom or disease of the subject would be effectively treated by a remyelination therapy. The remyelination therapy may be a particular remyelination therapy that uses a particular active ingredient.

In some embodiments, a method is provided that includes: determining, for each of electrode of a set of electrodes, a location on or in a body of a subject at which the electrode is or was positioned; detecting a set of evoked compound action potentials generated based on signals received by the set of electrodes while the set of electrodes are or were at the determined locations, wherein the set of evoked compound action potentials includes multiple subsets, and wherein each subset of the multiple subsets corresponds to a different electrode of the set of electrodes; for each subset of the multiple subsets of the set of evoked compound action potentials: mapping the subset to a functional system based on the place of the electrode that corresponds to the subset; generating an average evoked response using the subset of the subset of the set of evoked compound action potentials; extracting one or more features from the average evoked response; and generating a functional-system impairment metric associated with the functional system and the subject based on the one or more features, wherein the functional-system impairment metric indicates whether or an extent to which the functional system of the subject is impaired; generating a result based on the functional-system impairment metrics; and outputting the result.

Generating the result may include generating, for each subset of the multiple subsets, an impairment-change metric based on the functional-system impairment metric and based on another functional-system impairment metric associated with the functional system, the subject, and a previous time point. For each subset of at least one of the multiple subsets, generating the average evoked response may include aligning the subset of the set of evoked compound action potentials based on times at which preceding stimulations were delivered to the spinal cord of the subject. For each subset of at least one of the multiple subsets, generating the average evoked response may include aligning the subset of the set of evoked compound action potentials based on times at which preceding visual or auditory stimuli were presented to the subject. Generating the result may include determining that a treatment-adjustment criterion has been satisfied based on the functional-system impairment metrics, wherein the result includes a recommendation that a care provider consider a new treatment for the subject. Generating the result may include determining that a treatment-adjustment criterion has been satisfied based on the functional-system impairment metrics, and wherein the method further comprises prescribing a new treatment for the subject. The result includes an aggregation of the functional-system impairment metrics.

In some embodiments, a method is provided that includes: detecting a set of stimulation times at which a wearable or implanted stimulation device delivered a stimulation to a subject, wherein the wearable or implanted stimulation device is positioned to trigger or amplify nerve signals to facilitate partly or fully negating a disability of the subject; detecting a set of evoked compound action potentials that were measured using one or more electrodes, wherein each of the set of evoked compound action potentials is defined to start at a corresponding stimulation time of the set of stimulation times; generating an average evoked response using the set of evoked compound action potentials; extracting one or more features from the average evoked response; and generating a result based on the one or more features, wherein the result identifies: a predicted degree of inflammation; a predicted degree of myelination; a predicted degree of demyelination; or a predicted degree to which a symptom or disease of the subject would be effectively treated by a remyelination therapy.

The stimulation device may be a Functional Electrical Stimulation device that is configured and positioned to deliver stimulation to the paretic peroneal nerve. The stimulation device may be configured and positioned to deliver stimulation to the sacral nerve. The stimulation device may be configured and positioned to deliver stimulation to the cochlear nerve. The method may further include: automatically adjusting an intensity of stimulations that the stimulation device delivers based on the result and/or automatically adjusting a frequency of stimulations that the stimulation device delivers based on the result. The one or more features may include a time of a peak or trough in the average evoked response. The one or more features may include a magnitude or relative magnitude of a peak or trough in the average evoked response. Generating the result may include using a look-up table that associates various feature values with quantitative or qualitative predicted degrees of inflammation or of myelination. Generating the result may include using a function that relates feature values with quantitative or qualitative predicted degrees of inflammation or of myelination. The result may identify the predicted degree of inflammation. The result may identify the predicted degree of myelination or demyelination. The result may identify the predicted degree to which the symptom or disease of the subject would be effectively treated by a remyelination therapy.

In some embodiments, a system is provided that includes one or more data processors; and a non-transitory computer readable storage medium containing instructions which, when executed on the one or more data processors, cause the one or more data processors to perform part or all of one or more methods or processes disclosed herein. The system may further include a stimulation device configured to deliver stimulation pulses and/or a recording device configured to record evoked compound action potentials.

In some embodiments, a computer-program product tangibly embodied in a non-transitory machine-readable storage medium, including instructions configured to cause one or more data processors to perform part or all of one or more methods or processes disclosed herein.

In some embodiments, a system is provided that includes one or more means to perform part or all of one or more methods or processes disclosed herein.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1A illustrates how a stimulus can be delivered at a first stimulation site in the spinal record and evoked compound action potentials can be recorded at a different recording site.

FIG. 1B shows an exemplary average evoked response.

FIG. 2 illustrates a flowchart of a process of detecting and using evoked compound action potentials to predict a degree of myelination, demyelination, inflammation, or to which a symptom or disease would be effectively treated by given therapy.

FIG. 3 illustrates a flow chart of a process for generating impairment metrics for one or more functional systems of a subject.

FIG. 4 illustrates a flowchart of a process of detecting and using evoked compound action potentials to predict a degree of myelination, demyelination, inflammation, or to which a symptom or disease would be effectively treated by given therapy.

FIG. 5 illustrates exemplary positioning of electrodes.

FIG. 6 shows exemplary average evoked responses recorded at various sites in response to stimulation delivered one day after the stimulation electrodes were implanted.

FIG. 7 illustrates exemplary positioning of electrodes.

FIG. 8 shows exemplary average evoked responses recorded at various sites in response to stimulation delivered one week after the stimulation electrodes were implanted.

### DETAILED DESCRIPTION

As noted above, during exacerbations (i.e., relapses), a subject's immune cells attack myelin sheaths and oligodendrocytes, which leave axons vulnerable to degeneration that can cause neuronal death. Electrical impulses in the nervous system can travel from a first neuron's soma across the first neuron's axon to one or more other neurons' dendrites. Thus, axons support communication between cells. Even if the axon does not degenerate, the velocity of impulses traveling across the axon decreases if/when the myelin sheath surrounding the axon is damaged. This reduced impulse speed can produce functional deficits, as it may become difficult for the brain to quickly process stimuli, motor coordination, etc.

Recently, remyelination therapies (e.g., Clemastine Fumarate) are being explored. A remyelination therapy, if successful, may improve the speed of impulses traveling across axons, which may quickly provide improvement of symptomatic deficits. Further, if a remyelination therapy can trigger or support repair of a myelin sheath, degeneration of the axon and death of the neuron may be prevented. Not only may this reduce the extent to which a subject experiences new symptoms, but it may possibly delay or prevent relapse-remitting multiple sclerosis from transitioning into the neurodegenerative progressive stage.

However, currently, there are no biomarkers for measuring myelination (or demyelination or remyelination). For example, while magnetic resonance imaging (MRI) scans can show inflammation that is frequently interpreted as being demyelination, what is actually being captured are representations of the immune activity that underly the inflammation. As another example, evoked potentials (e.g., visual evoked potentials, somatosensory evoked potentials, motor evoked potentials, or brainstem auditory evoked potentials) may be used to diagnose or assess multiple sclerosis, given that the latency of evoked potentials is typically longer for multiple sclerosis subjects relative to healthy subjects (due to there being less myelin to support higher velocity impulses). However, assessments of evoked responses are constrained in that there is a limited spatial resolution at which the pathology can be characterized. Further, it is not sensitive or specific enough to detect local inflammation.

Thus, it would be useful to identify a sensitive neurophysiological biomarker of myelination (or remyelination or demyelination) that is relatively easy to measure to better inform pathology and treatment selection.

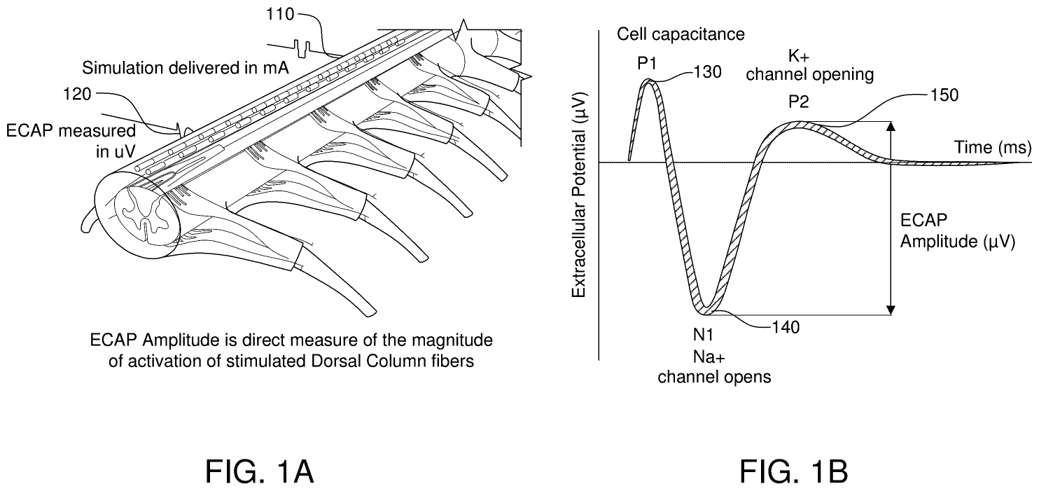

In some embodiments, a set of evoked compound action potentials are accessed. For example, FIG. 1A illustrates how a stimulus can be delivered at a first stimulation site 110 in the spinal record and evoked compound action potentials can be recorded at a different recording site 120. Stimulating the spinal cord may include eliciting an electrical stimulus via an electrode positioned percutaneously along the spinal cords. The stimulation can be elicited by an electrode that is part of a device implanted as part of a trial lead implant procedure (e.g., to determine whether and/or the extent to which stimulation of the spinal cord may alleviate pain or another symptom experienced by a subject).

The set of evoked compound action potentials may be recorded using a non-invasive electrode, such as an electroencephalography (EEG) electrode, or a minutely invasive electrode that does not require an incision, such as an electromyography (EMG) electrode. The set of evoked compound action potentials may obtained using one or more electrodes (e.g., non-invasive electrodes) positioned to record activity corresponding to a particular portion of the spinal cord (e.g., one or more particular vertebrae), one or more particular portions of the brain (e.g., particular cortices), or one or more particular muscles.

Depending on the locations of the electrodes, the evoked compound action potentials recorded in response to spinal-cord stimulation (e.g., invasive or minutely invasive spinal cord stimulation) can provide more precise data sets consisting of somatosensory cortical potentials, dromic nerve potentials, dromic compound muscle potentials, or evoked compound action potentials. The evoked compound action potentials represent activity that has a shorter latency and that does not involve synaptic transmission, unlike other types of evoked potentials that are currently used to assess various functional systems. Thus, analysis of evoked compound action potentials can provide a more specific measure of myelination.

An average evoked response can be generated by signals based on stimulation times for the set of evoked compound action potentials by signals based on and computing an average across the aligned signals. FIG. 1B shows an exemplary average evoked response. Particular features (e.g., local or global extrema values, times of the local or global extrema values, times of zero crossings, etc.) can be extracted and then used to generate a result that characterizes a state of a disease (e.g., multiple sclerosis), a degree of inflammation, a degree of myelination, or a prediction as to how well a given remyelination therapy (e.g., a particular remyelination therapy or a particular type of remyelination therapy) would treat the disease or a given symptom of the disease. In some instances, particular features can indicate or suggest mechanistic triggers or consequences of a disease state and/or myelination state. For example, a first peak 130 in the evoked compound action potential represents an increased capacitive current due to depolarization of the membranes of cells. A timing and magnitude of a first trough 140 corresponds to a timing and number of sodium channels opening. A timing and magnitude of a second peak 150 corresponds to a timing and number of potassium channels opening.

Thus, an average evoked response may be used to identify potential biomarkers for spinal cord demyelination in subjects with multiple sclerosis that may be present in evoked compound action potential signals and/or evoked muscle, peripheral, and/or cortical potentials. For a given subject, the biomarkers can then be used to predict an extent of demyelination. The predicted extent of demyelination can then be used (e.g., by itself or with other types of data) to predict an efficacy of a given treatment for the subject and/or to determine whether to recommend or provide a given treatment for the subject. The given treatment may include a remyelination treatment or a treatment for multiple sclerosis. An efficacy may be assessed with regard to a predicted impact that the treatment would have on a given type of system, a given functional system, remyelination, or stopping or slowing progression of multiple sclerosis. For example, it may be determined that a remyelination therapy is likely to improve motor deficits in subjects who have between 10-30% demyelination along a pathway corresponding to the cerebellar functional system.

Further or alternatively, features extracted from the average evoked response associated with a given subject may be used to assess a current disease state, to predict a disease prognosis and/or to predict an efficacy of a given treatment (e.g., multiple sclerosis treatment or remyelination treatment). This analysis may involve (for example) comparing the features to control data (e.g., associated with subjects that were not diagnosed with multiple sclerosis), assigning the feature data to a class (e.g., corresponding to other subjects with multiple sclerosis associated with a particular disease state, empirical progression, treatment responsiveness, etc.), etc. In some instances, a particular treatment can be recommended and/or provided to the subject based on the assessment of the current disease state, predicted disease prognosis and/or predicted treatment efficacy. For example, the particular treatment may include a multiple sclerosis treatment for which data has indicated a relatively high efficacy across other subjects associated with a similar disease state and/or prognosis as the given subject. As another example, the particular treatment may include a multiple sclerosis treatment for which the predicted efficacy for the subject was high (e.g., in an absolute sense or relative to other treatments).

It will be appreciated that processing of an average evoked response (that is based on the evoked compound action potentials) may further include processing other data, such as other multimodal evoked potentials.

It will further be appreciated that an approach that uses an electrode from a device in a trial implant procedure to deliver the stimulation therefore also repurposes the device (e.g., to facilitate detecting areas of (re)myelination or demyelination or—as further explained herein—to facilitate generating a diagnosis, prognosis, or treatment recommendation).

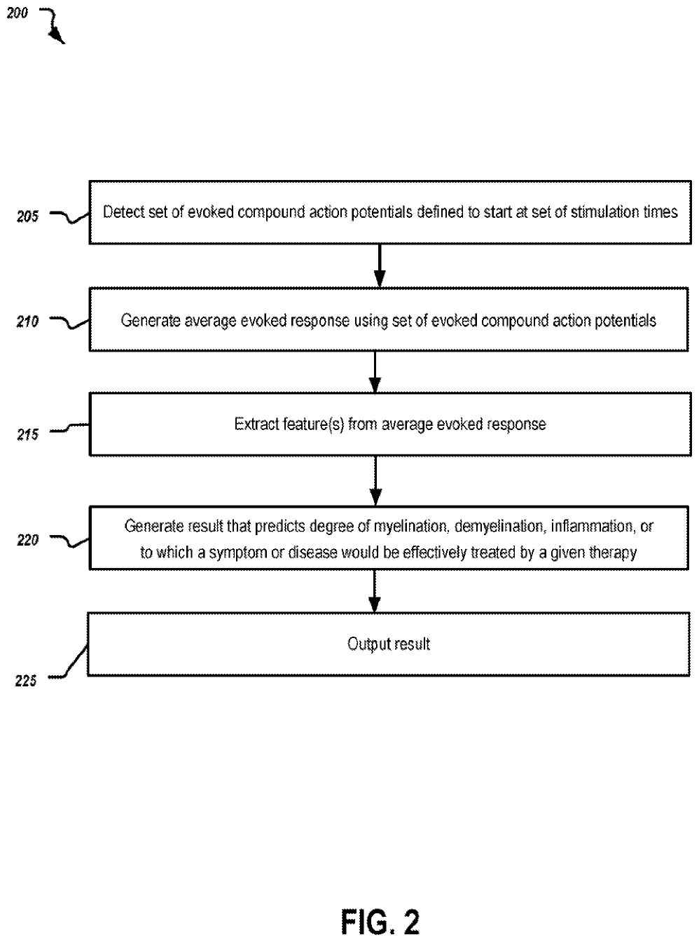

FIG. 2 illustrates a flowchart of a process 200 of detecting and using evoked compound action potentials to predict a degree of myelination, demyelination, inflammation, or to which a symptom or disease would be effectively treated by given therapy. Process 200 begins at block 205, where a set of evoked compound action potentials are detected. For example, an electrode (e.g., a non-invasive or minutely invasive electrode) can be positioned to record voltage signals from a part of the spinal cord, a part of the brain, a muscle, etc. of a given subject. While the voltage signals may be continuously received, they can be processed to extract particular signal portions that correspond to time windows that begin at times that stimulation was delivered (e.g., to the spinal cord). The stimulations may have been delivered using an electrode positioned via percutaneous access to the spinal cord. In some instances, the stimulations are delivered using a lead in a device implanted as part of a trial lead implant procedure (e.g., to determine whether or an extent to which spinal-cord stimulation alleviates pain or another symptom of a subject). The stimulation(s) may, in fact, be the same stimulations that are delivered as part of the trial lead implant procedure, meaning that the device and operation of the device can have multiple purposes. The set of evoked compound action potentials may include a dromic compound action potential and/or an antidromic compound action potential.

At block 210, an average evoked response is generated using the set of evoked compound action potentials. The average evoked response may be defined to be an average of the set of evoked compound action potentials collected from a given electrode or a given set of electrodes positioned to record activity from a particular area of the body. It will be appreciated that, in some instances, different electrodes (or different sets of electrodes) are positioned to record activity from different areas of the body, which can be processed to assess different pathways and/or functional systems.

At block 215, one or more features are extracted from the average evoked response. A feature may include (for example) a magnitude of a peak, a magnitude of a trough, a time of a peak, a time of a trough, a time of a zero crossing, etc. A magnitude of a peak or trough may be absolute or relative (e.g., to a magnitude of another peak or trough). A time of a peak, trough or zero crossing may be absolute or may be a time difference (e.g., relative to another peak, trough of zero crossing). A feature may include a principal component or a kernel. A feature may include a weighted sum of multiple characteristics (e.g., magnitudes, times, etc.) of the average evoked response. In some instances, a machine-learning model (e.g., a regression model, model based on one or more decision trees, neural network, etc.) is used to identify features that are predictive of a given variable (e.g., a current or subsequent EDSS score, a current or subsequent lesion load, an extent to which a remyelination treatment or a multiple sclerosis treatment improves a motor capability of a subject, an extent to which a remyelination treatment or a multiple sclerosis treatment improves a non-motor function of a subject, etc.).

At block 220, a result is generated based on the feature(s), where the result corresponds to a prediction of a degree of myelination, a degree of demyelination, a degree of inflammation, or a degree to which a symptom or disease of the subject would be effectively treated by a given therapy. The result may be a number on a scale (e.g., where values at one end of the scale represent myelination levels of healthy individuals or no inflammation and where values at the other end of the scale represent myelination levels, complete demyelination, complete disruption of a pathway, or maximum inflammation). Thus, absolute values of the result may lack meaning but rather, the result may be meaningful in that it can provide a basis for comparison across time and/or across subjects.

The result may be a scaled version (e.g., normalized version) of a feature or a weighted sum of multiple features. The result may be generated by comparing each of one or more features to one or more corresponding thresholds. For example, for each feature, it may be determined which one of five ranges (defined for the type of feature) includes the feature. A number of points may be assigned based on the range, and the feature-specific points may be added together to generate the result.

In addition to or instead of the result being numerical, the result may include a category. For example, a numeric value (e.g., a numeric feature value or a numeric value generated based on one or more feature values) may be assigned to one of multiple ranges, where each range is associated with a distinct category. A category may represent predicted degree of myelination, a predicted degree of demyelination, a predicted degree of inflammation, a predicted degree to which a symptom or disease would be effectively treated by a given therapy. A category may represent whether and/or a degree to which a given clinical study or therapy is recommended.

At block 225, the result is output. For example, the result may be transmitted to or presented at a user device (e.g., associated with a care provider or coordinator of a clinical study).

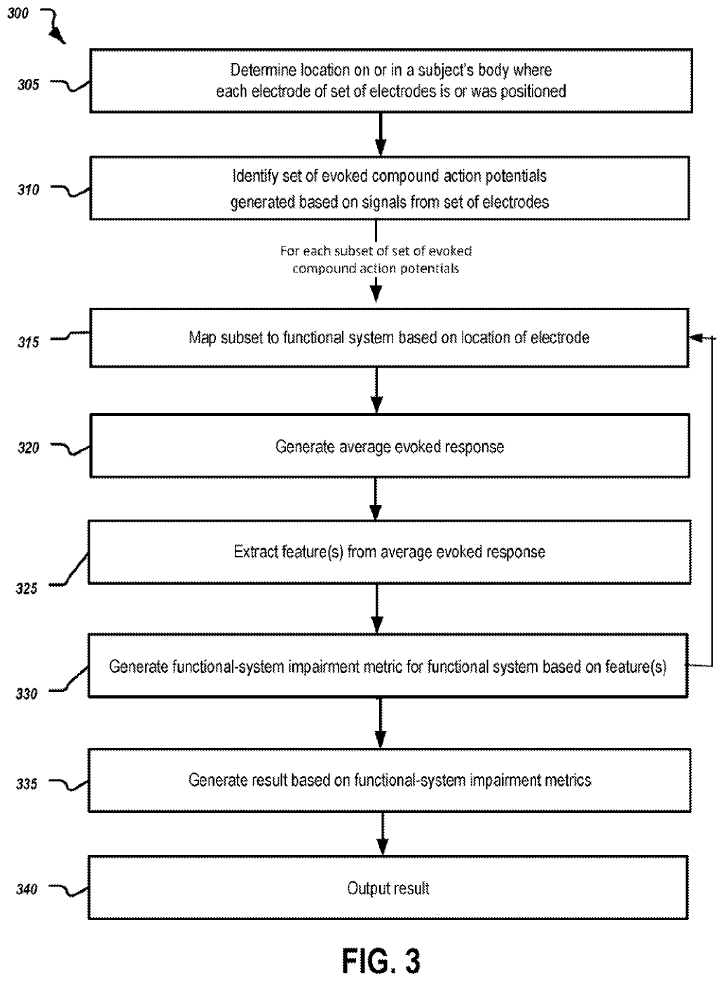

FIG. 3 illustrates a flow chart of a process 300 for generating impairment metrics for one or more functional systems of a subject. Process 300 begins at block 305 where it is determined where, on a subject's body, an electrode of a set of electrodes is or was placed. The set of electrodes may include one or more electroencephalography electrodes and/or one or more electromyography electrodes. Block 305 may include identifying over which brain region, over which part of the spinal cord, over which muscle, or in which muscle the electrode was positioned. For example, block 305 may include determining that a given electrode is positioned over a particular cortical region (e.g., visual cortex, motor cortex, etc.), brainstorm, cerebellum, vertebrae, etc.

In some instances, it is assumed that the subject is or was wearing a particular type of EEG cap that includes electrodes at known relative positions and is to be worn in a particular orientation. Placements of the electrodes may then be assumed. In some instances, instructions may have been provided to the subject or a care provider as to where to position the electrode(s), and it can then be assumed the electrodes were positioned accordingly.

At block 310, a set of evoked compound action potentials are identified, where the set of evoked compound action potentials were generated based on signals received by the set of electrodes. The set of evoked compound action potentials may include evoked compound action potentials generated based on signals collected by an electrode (e.g., a non-invasive or minutely invasive electrode), which may have been positioned to record voltage signals from a part of the spinal cord, a part of the brain, a muscle, etc. of a given subject. While the voltage signals may be continuously received, they can be processed to extract particular signal portions that correspond to time windows that begin at times that stimulation was delivered, and each evoked compound action potential may be defined to be an average of the evoked aligned responses corresponding to a given recording site. The stimulation may be (for example) an electrical stimulation (e.g., of the spinal cord), visual stimulation (e.g., a dynamically changing white-and-black checkboard), or an auditory stimulation.

Blocks 315-330 can be repeated for each subset of the set of evoked compound action potentials. Each subset can correspond to a distinct recording site, a distinct functional system, a distinct recording electrode and/or a distinct group of recording electrodes.

At block 315, the subset of the set of evoked compound action potentials can be mapped to a functional system based on the location of the corresponding electrode(s) (that collected the signals corresponding to the subset). The mapping may be performed using a look-up table.

At block 320, an average evoked response can be generated. The average evoked response can be generated using the set of evoked compound action potentials. The average evoked response may be defined to be an average of the set of evoked compound action potentials collected from a given electrode or a given set of electrodes positioned to record activity from a particular area of the body.

At block 325, one or more features are extracted from the evoked response. A feature may include (for example) a magnitude of a peak, a magnitude of a trough, a time of a peak, a time of a trough, a time of a zero crossing, etc. A magnitude of a peak or trough may be absolute or relative (e.g., to a magnitude of another peak or trough). A time of a peak, trough or zero crossing may be absolute or may be a time difference (e.g., relative to another peak, trough of zero crossing). A feature may include a principal component or a kernel. A feature may include a weighted sum of multiple characteristics (e.g., magnitudes, times, etc.) of the average evoked response. In some instances, a machine-learning model (e.g., a regression model, model based on one or more decision trees, neural network, etc.) is used to identify features that are predictive of a given variable (e.g., a current or future functional-system score as defined in accordance with the EDSS specifications). It will be appreciated that the feature(s) extracted during an assessment of one functional system need not be the same as the feature(s) extracted during an assessment of another functional system.

At block 330, a functional-system impairment metric can be generated for the functional system. The functional-system impairment metric may be (for example) a numeric score along a scale (e.g., a scale that extends from 0 to 9) or a category. In some instances, the functional-system impairment metric is a predicted value of a Kurtzke Function System Score. In some embodiments, the functional-system impairment metric is determined using only the extracted feature(s), whereas in other embodiments, the functional-system impairment metric is determined using the extracted feature(s) and other types of data (e.g., data from an accelerometer, which may give an indication of mobility and/or tremors).

Blocks 315-330 can be repeated for each of two or more functional systems (e.g., pyramidal, cerebellar, brainstem, visual, and/or cerebral).

At block 335, a result is generated based on the functional-system impairment metrics. The result may be (for example) an aggregation (e.g., list or vector), a sum, a weighted sum, an average, or a maximum of the functional-system impairment metrics. A result may further or alternatively include a comparison or difference between each of the functional-system impairment metrics and corresponding older functional-system impairment metrics previously determined based on evoked compound action potentials for the subject.

At block 340, the result is output. For example, the result may be transmitted to or presented at a user device (e.g., associated with a care provider or coordinator of a clinical study). In some instances, process 300 includes evaluating an alert criterion based on the result (e.g., to determine whether a functional-system impairment metric has exceeded a threshold, whether a numeric result (e.g., a sum, a weighted sum, an average, or a maximum of the functional-system impairment metrics) has exceeded a threshold, whether a difference between a current and previous functional-system impairment metric has exceeded a threshold, or to determine whether a difference between a current and previous numeric result has exceeded a threshold. Block 340 may then be selectively performed when the condition is satisfied, or a type or destination of output may be selected based on whether the condition is satisfied. For example, the result may be transmitted to a device that updates subject records when the condition is not satisfied and may be transmitted to an email server of a care provider when the condition is satisfied (to alert the care provider). As another example, the result may include a recommendation that a care provider consider a new treatment for the subject when the condition is satisfied.

It will be appreciated that process 300 may provide an opportunity to provide an image, map and/or location-specific information that predicts where demyelination (or remyelination) has occurred. For example, if a latency between a time zero (stimulation time) and a time of a given peak in the average evoked response is longer (relative to a corresponding healthy-subject control) for a first functional system than is a latency time zero (stimulation time) and a time of a given peak in another average evoked response is longer (relative to another corresponding healthy-subject control) for a second functional system, it may be inferred that there is more demyelination in the path corresponding to the first functional system than the second functional system. (A normalization may also be applied to account for a length between a stimulation site and recording sites corresponding to the two functional systems.) As another example, locations of various recording electrodes (e.g., and of one or more stimulating electrodes) can be used to generate an image that illustrates where in a subject's body the nervous system is predicted to be demyelinated (e.g., an extent of the demyelination). This spatial information may be inferred based on (for example) differences between the extent to which latencies of peaks in average evoked responses are delayed and/or amplitudes of peaks in average evoked responses are reduced relative to corresponding controls and then comparing the degree of delay or amplitude reduction across different recording sites. To illustrate, consider an instance where a stimulating lead is implanted between thoracic vertebrae 7 and 8, a first recording electrode is positioned over cervical vertebrae 2, and a second recording electrode is positioned over the frontal lobe. If there is no delay or reduced magnitude in one or more peaks of a first average evoked response generated based on signals from the first recording electrode (relative to a first control) but there is for a second average evoked response generated based on signals from the second recording electrode (relative to a second control), it can be inferred that there is full myelination between vertebrae 7 and vertebrae 2, but that there is some demyelination in a pathway between vertebrae 2 and the frontal lobe.

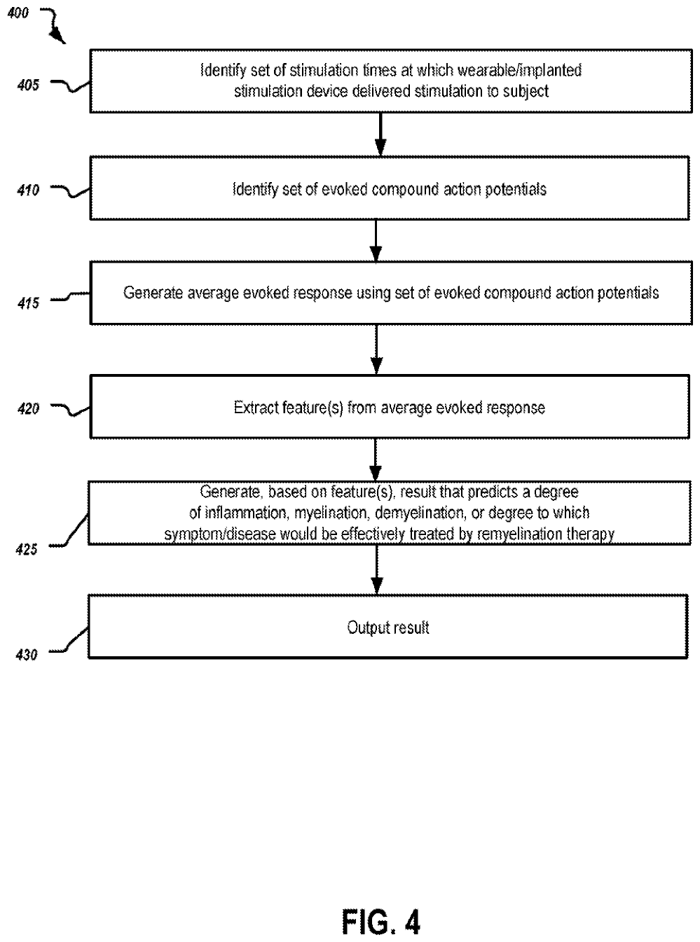

FIG. 4 illustrates a flowchart of a process 400 of detecting and using evoked compound action potentials to predict a degree of myelination, demyelination, inflammation, or to which a symptom or disease would be effectively treated by given therapy. Process 400 begins at block 405, where a set of stimulation times are identified, each of the stimulation times being a time at which a wearable or implanted device elicited a stimulation. The wearable or implanted device may have been positioned to trigger or amplify nerve signals to facilitate partly or fully negating a disability of the subject. The disability may be one that was caused by multiple sclerosis. For example, the device may be a Functional Electrical Stimulation device that is configured and positioned to deliver stimulation to the paretic peroneal nerve (e.g., to reduce or eliminate a drop-foot symptom). As another example, the stimulation device may be configured and positioned to deliver stimulate the sacral nerve to reduce incontinence. As yet another example, the stimulation device may be configured and positioned to stimulate the auditory nerve to facilitate transmitting signals from the peripheral nervous system to the central nervous system.

At block 410, a set of evoked compound action potentials are detected. For example, an electrode (e.g., a non-invasive or minutely invasive electrode) can be positioned to record voltage signals from a part of the spinal cord, a part of the brain, a muscle, etc. of a given subject. While the voltage signals may be continuously received, they can be processed to extract particular signal portions that correspond to time windows that begin at times that stimulation was delivered (e.g., to the spinal cord). The set of evoked compound action potentials may include a dromic compound action potential and/or an antidromic compound action potential.

At block 415, an average evoked response is generated using the set of evoked compound action potentials. The average evoked response may be defined to be an average of the set of evoked compound action potentials collected from a given electrode or a given set of electrodes positioned to record activity from a particular area of the body.

At block 420, one or more features are extracted from the average evoked response. A feature may include (for example) a magnitude of a peak, a magnitude of a trough, a time of a peak, a time of a trough, a time of a zero crossing, etc. A magnitude of a peak or trough may be absolute or relative (e.g., to a magnitude of another peak or trough). A time of a peak, trough or zero crossing may be absolute or may be a time difference (e.g., relative to another peak, trough of zero crossing). A feature may include a principal component or a kernel. A feature may include a weighted sum of multiple characteristics (e.g., magnitudes, times, etc.) of the average evoked response.

In some instances, a machine-learning model (e.g., a regression model, model based on one or more decision trees, neural network, etc.) is used to identify features that are predictive of a given variable (e.g., a current or subsequent Functional System Score, a current or subsequent function-system impairment metric, a current or subsequent EDSS score, a current or subsequent lesion load, an extent to which a remyelination treatment or a multiple sclerosis treatment improves a motor capability of a subject, an extent to which a remyelination treatment or a multiple sclerosis treatment improves a non-motor function of a subject, etc.). In some instances, the machine-learning model is trained to identify features that are predictive of a magnitude of a particular disability or symptom (e.g., multiple sclerosis symptom).

At block 425, a result is generated based on the feature(s), where the result corresponds to a prediction of a degree of myelination, a degree of demyelination, a degree of inflammation, or a degree to which a symptom or disease of the subject would be effectively treated by a given therapy. The result may be a number on a scale (e.g., where values at one end of the scale represent myelination levels of healthy individuals or no inflammation and where values at the other end of the scale represent myelination levels complete demyelination, complete disruption of a pathway, or maximum inflammation). Thus, absolute values of the result may lack meaning but rather, the result may be meaningful in that it can provide a basis for comparison across time and/or across subjects.

The result may be a scaled version (e.g., normalized version) of a feature or a weighted sum of multiple features. The result may be generated by processing the feature(s) using a function. The function may include (for example) a monotonic or non-monotonic function, a stepwise function, a probabilistic function, etc.

In some instances, the result may be a parameter for the stimulation device. For example, the result may identify a magnitude of a stimulation, a frequency of a stimulation, a temporal pattern of a stimulation, a threshold for stimulation, etc. For example, the feature(s) may be processed using a function to generate a result that indicates a recommended magnitude of stimulation.

The result may be categorical or numerical.

In some instances, the result characterizes or is a predicted functional-system impairment metric, a predicted functional system score, a predicted degree of myelination, a predicted degree of demyelination, a predicted degree of inflammation, a predicted degree to which a symptom or disease would be effectively treated by a given therapy. A category may represent whether and/or a degree to which a given clinical study or therapy is recommended.

At block 430, the result is output. For example, the result may be transmitted to or presented at a user device (e.g., associated with a care provider or the subject).

### EXAMPLES

Approximately 3% of people diagnosed with multiple sclerosis have had a spinal cord stimulator implanted for chronic pain, as have other people without multiple sclerosis. In Examples 1 and 2, a separate stimulation device was connected to the implanted percutaneous spinal cord electrode leads. The separate spinal cord stimulation device was then used to stimulate the spinal cord, and evoked compound action potentials were recorded across multiple points in the central nervous system.

### Example 1

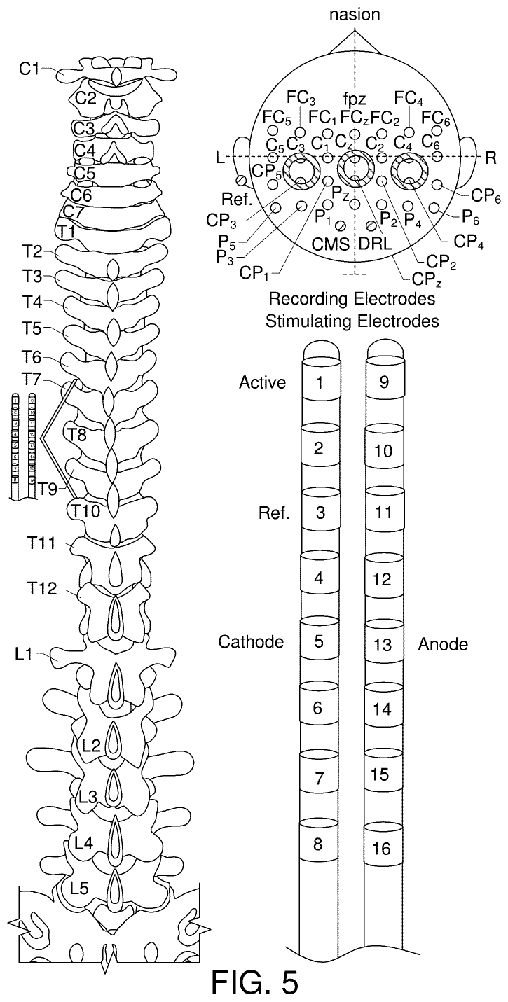

As shown in FIG. 5, anode and cathode stimulating electrodes (contacts 5 and 13 on the spinal cord electrode leads) were positioned near thoracic vertebrae 7 and 8. Contacts 1 and 3 on the spinal cord leads served as the recording electrodes (active and reference electrodes, respectively). Further, cortical signals were recorded from the CP3, CPz and CP4 sites using gold cup electrodes placed on the subject's scalp, and electromyography electrodes were used to record motor responses from the subject's legs. The electrical stimulation delivered by the stimulating electrodes was defined to be 500 pulses, 15000 uA at 4.55 Hz. Average evoked responses were generated for each recording site by extracting windows of recorded activity which began from the time of stimulation onset.

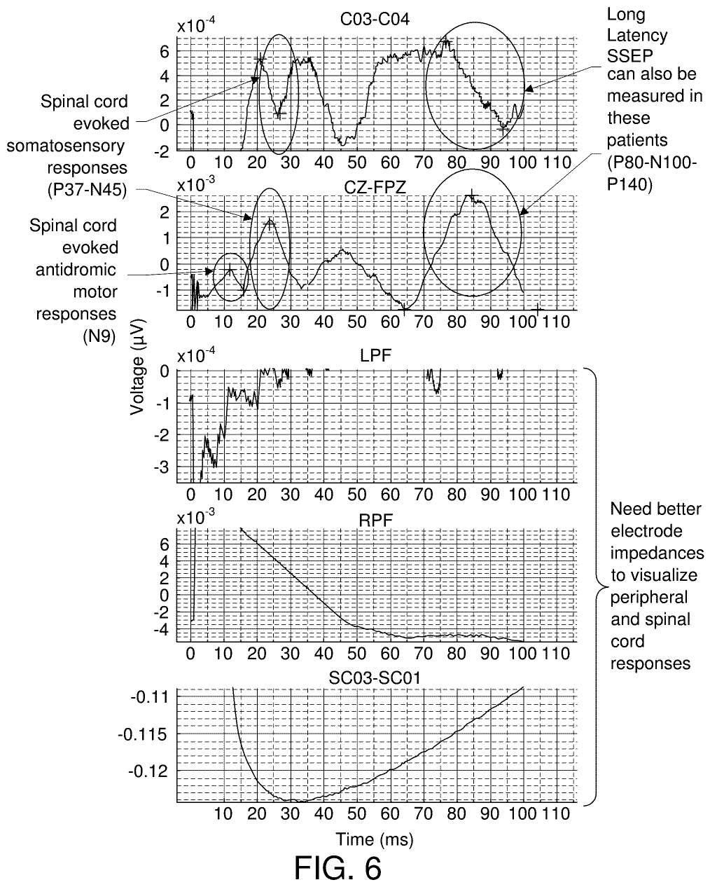

FIG. 6 shows average evoked responses recorded at various sites in response to stimulation delivered one day after the stimulation electrodes were implanted. The top graph shows the average evoked cortical response recorded using active and reference electrodes positioned at Cp3 and Cp4 on the scalp(as shown in FIG. 5). The second to top graph shows the average cortical evoked response recorded at the Cpz site referenced to Fpz. The third and fourth graphs show the average evoked peripheral responses recorded from the left and right popliteal fossa located on the left and right leg. The bottom graph shows the average evoked compound action potential responses recorded using the e 1st and 9th contacts on percutaneous spinal cord leads implanted in the spinal cord at thoracic vertebrae 7-8. A sharp response is observable shortly after t=0 across the top four curves, and this is an artifact due to the stimulation. However, subsequent peaks correspond to physiological responses to the stimulation. For example, the second graph in FIG. 6 shows a first positive peak around 20 ms after the stimulation and a negative peak around 25 ms after the stimulation. It can be inferred based on the latency of the responses that the first peak corresponds to spinal cord evoked antidromic motor responses (N9) and that the second peak corresponds to spinal cord evoked somatosensory responses (P37-N45). The later peak observed around 85 ms after the stimulation indicates that long latency somatosensory evoked potentials can also be measured.

The third through fifth average evoked responses lack clear peaks. Imaging that was subsequently performed showed that leads had crossed, which likely led to inadequate recordings.

### Example 2

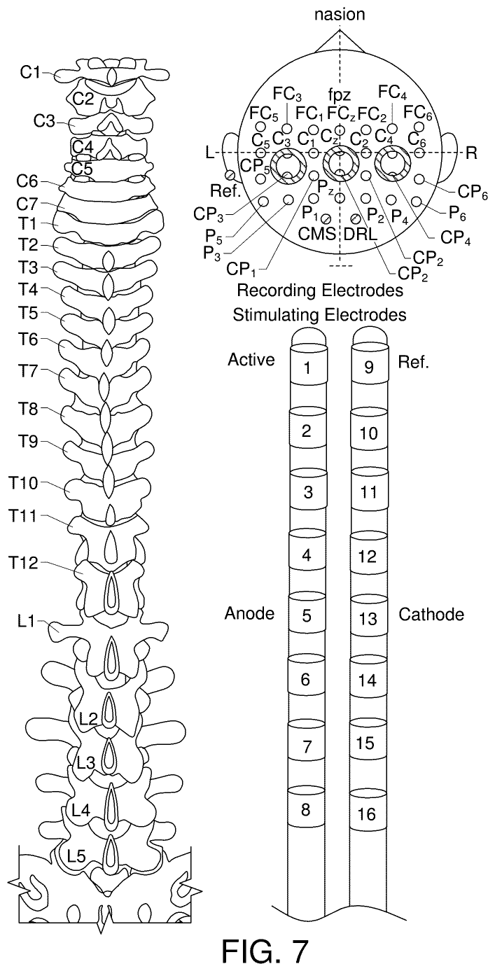

Another set of recordings was completed in the same patient one week following lead implantation. As illustrated in FIG. 7, the same contacts, 5 and 13, were used to record from the thoracic spinal cord. Cortical signals were again recorded from the CP3, CPz and CP4 using gold cup electrodes placed on the scalp and electromyography electrodes were used to record motor responses from the subject's leg. The stimulation delivered through stimulating electrodes was defined to be 500 pulses, 15000 uA at 4.55 Hz. Average evoked responses were generated for each recording site by extracting, from evoked compound action potentials, windows of recorded activity that begin at the stimulation times and averaging the windows.

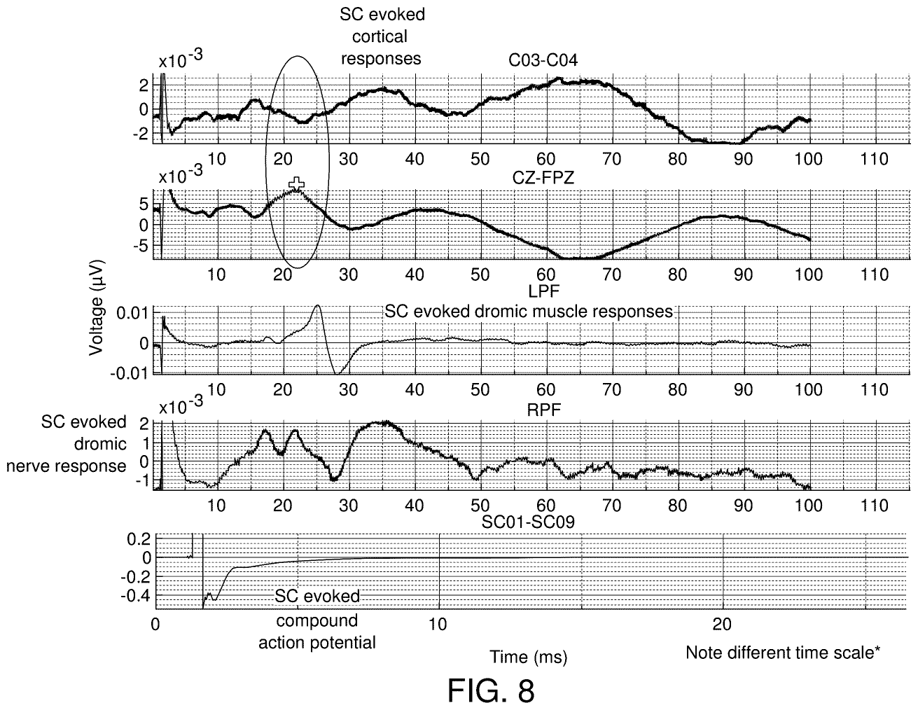

FIG. 8 shows average evoked responses recorded at various sites in response to stimulation delivered one week after the stimulation electrodes were implanted. The top graph shows the average evoked cortical response recorded using active and reference electrodes positioned at the Cp3 and Cp4 locations on the scalp. The second to top graph shows the average evoked cortical response recorded at the CPz site referenced to Fpz. The third and fourth graphs show the average evoked responses recorded at peripheral electrodes positioned over the left and right popliteal fossa. The bottom graph shows the average evoked compound action potential recorded using the 1st and 9th contacts on the percutaneous, implanted spinal cord electrode leads (as shown in FIG. 7). (The time scale of the bottom curve differs from that of the other.)

A sharp response is observable shortly after t=0 across all curves, and this is an artifact due to the stimulation. However, subsequent peaks correspond to physiological responses to the stimulation. For example, the second graph in FIG. 8 again shows a first positive peak around 20 ms after the stimulation, a negative peak around 25 ms after the stimulation, and a later peak is observed around 85 ms after the stimulation. It can again be inferred based on latencies of observed peaks that the first peak corresponds to spinal cord evoked antidromic motor responses (N9), that the second peak corresponds to spinal cord evoked somatosensory responses (P37-N45), and that the third peak corresponds to long latency somatosensory evoked responses.

In this instance, the leads were not crossed, so the third and fourth curves are interpretable. Specifically, the third and fourth curves include clear peaks which show a compound nerve action potential (20 ms, third curve) and an evoked compound action potential (2-3 ms, fourth curve).

Therefore, the times and/or magnitudes of various peaks and/or troughs of one or more average evoked responses may be determined for a given subject and used to infer (for example) a degree of myelination, a degree of demyelination, a degree of inflammation, a degree to which a given treatment is predicted to be effective in treating the subject, etc. Further or alternatively, the times and/or magnitudes of various peaks and/or troughs of one or more average evoked responses may be used to generate a functional-system impairment metric for each of one or more functional systems.

These examples indicate that average evoked responses may support a biomarker for a state of a subject's disease, a prognosis, or a degree to which a subject's disease would effectively respond to a given treatment. Further, average evoked responses may provide an avenue for assessing clinical and subclinical demyelination. Finally, average evoked responses may be useful for measuring how effective a treatment is in preserving axons and/or in triggering remyelination.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The present description provides preferred exemplary embodiments only, and is not intended to limit the scope, applicability or configuration of the disclosure. Rather, the present description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the present description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A method comprising:
detecting a set of evoked compound action potentials that were measured using one or more electrodes, wherein each of the set of evoked compound action potentials is defined to start at a corresponding stimulation time of a set of stimulation times;
generating an average evoked response using the set of evoked compound action potentials;
extracting one or more features from the average evoked response;
generating a result based on the one or more features, wherein the result identifies:
a predicted degree of inflammation;
a predicted degree of myelination;
a predicted degree of demyelination; or
a predicted degree to which a symptom or disease of the subject would be effectively treated by a remyelination therapy; and
outputting the result.

2. The method of claim 1, wherein each of the set of evoked compound action potentials includes an antidromic compound action potential.

3. The method of claim 1, wherein each of the set of evoked compound action potentials includes a dromic compound action potential.

4. The method of claim 1, wherein the spinal cord was stimulated at each of the set of stimulation times using an electrode positioned via percutaneous access to the spinal cord.

5. The method of claim 1, wherein the one or more features includes a time of a peak or trough in the average evoked response.

6. The method of claim 1, wherein the one or more features includes a magnitude or relative magnitude of a peak or trough in the average evoked response.

7. The method of claim 1, wherein the one or more electrodes were positioned on a head of the subject to collect the set of evoked compound action potentials using signals from a brain of the subject.

8. The method of claim 1, wherein generating the result includes using a look-up table that associates various feature values with quantitative or qualitative predicted degrees of inflammation or of myelination.

9. The method of claim 1, wherein generating the result includes using a function that relates feature values with quantitative or qualitative predicted degrees of inflammation or of myelination.

10. The method of claim 1, wherein the result is a category.

11. The method of claim 1, further comprising:
stimulating the spinal cord of the subject using the one or more electrodes at each of the set of stimulation times; and
measuring the set of evoked compound action potentials.

12. The method of claim 1, wherein the result identifies the predicted degree of inflammation.

13. The method of claim 1, wherein the result identifies the predicted degree of myelination or demyelination.

14. The method of claim 1, wherein the result identifies the predicted degree to which the symptom or disease of the subject would be effectively treated by a remyelination therapy.

15. The method of claim 1, wherein the remyelination therapy is a particular remyelination therapy that uses a particular active ingredient.

16. A method comprising:
determining, for each of electrode of a set of electrodes, a location on or in a body of a subject at which the electrode is or was positioned;
detecting a set of evoked compound action potentials generated based on signals received by the set of electrodes while the set of electrodes are or were at the determined locations, wherein the set of evoked compound action potentials includes multiple subsets, and wherein each subset of the multiple subsets corresponds to a different electrode of the set of electrodes;
for each subset of the multiple subsets of the set of evoked compound action potentials:
mapping the subset to a functional system based on the place of the electrode that corresponds to the subset;
generating an average evoked response using the subset of the subset of the set of evoked compound action potentials;
extracting one or more features from the average evoked response; and
generating a functional-system impairment metric associated with the functional system and the subject based on the one or more features, wherein the functional-system impairment metric indicates whether or an extent to which the functional system of the subject is impaired;
generating a result based on the functional-system impairment metrics; and
outputting the result.

17. The method of claim 16, wherein generating the result includes:
generating, for each subset of the multiple subsets, an impairment-change metric based on the functional-system impairment metric and based on another functional-system impairment metric associated with the functional system, the subject, and a previous time point.

18. The method of claim 16, wherein, for each subset of at least one of the multiple subsets, generating the average evoked response includes aligning the subset of the set of evoked compound action potentials based on times at which preceding stimulations were delivered to the spinal cord of the subject.

19. The method of claim 16, wherein, for each subset of at least one of the multiple subsets, generating the average evoked response includes aligning the subset of the set of evoked compound action potentials based on times at which preceding visual or auditory stimuli were presented to the subject.

20. The method of claim 16, wherein generating the result includes determining that a treatment-adjustment criterion has been satisfied based on the functional-system impairment metrics, wherein the result includes a recommendation that a care provider consider a new treatment for the subject.

21. The method of claim 16, wherein generating the result includes determining that a treatment-adjustment criterion has been satisfied based on the functional-system impairment metrics, and wherein the method further comprises prescribing a new treatment for the subject.

22. The method of claim 16, wherein the result includes an aggregation of the functional-system impairment metrics.

23. A method comprising:
detecting a set of stimulation times at which a wearable or implanted stimulation device delivered a stimulation to a subject, wherein the wearable or implanted stimulation device is positioned to trigger or amplify nerve signals to facilitate partly or fully negating a disability of the subject;
detecting a set of evoked compound action potentials that were measured using one or more electrodes, wherein each of the set of evoked compound action potentials is defined to start at a corresponding stimulation time of the set of stimulation times;
generating an average evoked response using the set of evoked compound action potentials;
extracting one or more features from the average evoked response; and
generating a result based on the one or more features, wherein the result identifies:
a predicted degree of inflammation;
a predicted degree of myelination;
a predicted degree of demyelination; or
a predicted degree to which a symptom or disease of the subject would be effectively treated by a remyelination therapy.

24. The method of claim 23, wherein the stimulation device is a Functional Electrical Stimulation device that is configured and positioned to deliver stimulation to the paretic peroneal nerve.

25. The method of claim 23, wherein the stimulation device is configured and positioned to deliver stimulation to the sacral nerve.

26. The method of claim 23, wherein the stimulation device is configured and positioned to deliver stimulation to the cochlear nerve.

27. The method of claim 23, further comprising: automatically adjusting an intensity of stimulations that the stimulation device delivers based on the result.

28. The method of claim 23, further comprising: automatically adjusting a frequency of stimulations that the stimulation device delivers based on the result.

29. The method of claim 23, wherein the one or more features includes a time of a peak or trough in the average evoked response.

30. The method of claim 23, wherein the one or more features includes a magnitude or relative magnitude of a peak or trough in the average evoked response.

31. The method of claim 23, wherein generating the result includes using a look-up table that associates various feature values with quantitative or qualitative predicted degrees of inflammation or of myelination.

32. The method of claim 23, wherein generating the result includes using a function that relates feature values with quantitative or qualitative predicted degrees of inflammation or of myelination.

33. The method of claim 23, wherein the result identifies the predicted degree of inflammation.

34. The method of claim 23, wherein the result identifies the predicted degree of myelination or demyelination.

35. The method of claim 23, wherein the result identifies the predicted degree to which the symptom or disease of the subject would be effectively treated by a remyelination therapy.

36. A system comprising:
one or more data processors; and
a non-transitory computer readable storage medium containing instructions which, when executed on the one or more data processors, cause the one or more data processors to perform a set of actions including:
detecting a set of evoked compound action potentials that were measured using one or more electrodes, wherein each of the set of evoked compound action potentials is defined to start at a corresponding stimulation time of a set of stimulation times;
generating an average evoked response using the set of evoked compound action potentials;
extracting one or more features from the average evoked response;
generating a result based on the one or more features, wherein the result identifies:
a predicted degree of inflammation;
a predicted degree of myelination;
a predicted degree of demyelination; or
a predicted degree to which a symptom or disease of the subject would be effectively treated by a remyelination therapy; and
outputting the result.

37. The system of claim 36, further comprising a stimulation device configured to deliver stimulation pulses.

38. The system of claim 36, further comprising a recording device configured to record evoked compound action potentials.

39. A computer-program product tangibly embodied in a non-transitory machine-readable storage medium, including instructions configured to cause one or more data processors to perform a set of actions including:
detecting a set of evoked compound action potentials that were measured using one or more electrodes, wherein each of the set of evoked compound action potentials is defined to start at a corresponding stimulation time of a set of stimulation times;
generating an average evoked response using the set of evoked compound action potentials;
extracting one or more features from the average evoked response;
generating a result based on the one or more features, wherein the result identifies:
a predicted degree of inflammation;
a predicted degree of myelination;
a predicted degree of demyelination; or
a predicted degree to which a symptom or disease of the subject would be effectively treated by a remyelination therapy; and
outputting the result.
